(19) United States
(12) Patent Application Publication
(10) Pub. No.: US 2009/0202119 A1
(43) Pub. Date: Aug. 13, 2009

(54) Method for analyzing and processing fluorescent images

(76) Inventors: Martin Hefti, Lenzburg (CH); Herbert Looser, Windisch (CH); Hans Landolt, Aarau (CH)

Correspondence Address: Martin HEFTI, Promenade 8, CH-5600 Lenzburg, CH

(21) Appl. No.: 12/316,810

(22) Filed: Dec. 15, 2008

(30) Foreign Application Priority Data
Dec. 19, 2007 (CH) 01969/07

(51) Int. Cl. G06K 9/00; A61B 6/00

(52) U.S. Cl. 382/128; 600/476

(57) ABSTRACT

A method for analysis and processing of fluorescent images. Fluorescent light is emitted by tumor tissue in an illuminated surgical area detected by an image detection device and is forwarded to an image processing system. After determination of a maximal intensity, the image processing system determines a threshold value as a predefined fraction of the maximal intensity. With basic morphological operations of image processing, limit intensity lines which separate the tumor tissue of intensities above the threshold value and normal tissue of intensities below the threshold value are generated and imaged in a line profile image, wherein the line profile image can be superimposed on the fluorescent image.

### BACKGROUND OF THE INVENTION

1. Field of the Invention

This invention describes a method for analyzing and processing fluorescent images of a medical surgical microscope.

2. Discussion of Related Art

The fluorescence of tumor tissue, which is achieved by enrichment of the tumor tissue with special contrast media and by subsequent illumination with UV light, has been used in medicine for a long time, such as to support resection of portions of tissue in the operating room.

In this method, known as intraoperative fluorescence detection, a natural endogenous substance (5-aminolevulinic acid, abbreviated 5-ALA), is administered to a patient with a gliobastoma before operating on the brain tumor. 5-ALA is formed in the body as part of the synthesis of hemoglobin, the pigment in blood, and is indispensable in hematopoiesis. It has the property of being converted to the pigment protoporphyrin IX in malignant brain tumor tissue. Protoporphyrin IX accumulates in neoplastic cells and therefore especially in tumor tissue. Protoporphyrin IX has a typical fluorescence at a wavelength of approx. 635 nm when irradiated with UV light. Protoporphyrin IX molecules absorb the exciting UV light and then emit a longer-wavelength fluorescent light of a lower energy, so that the tumor tissue has a red fluorescence.

The tumor to be removed often has a wide cell border infiltrating the normal tissue, or tumor cell clusters are surrounded by normal tissue, so it is difficult to remove only the tumor cells with minimal damage to the normal tissue. Although the surgeon sees a red fluorescence in the fluorescent image, the surgeon must evaluate independently how much tissue is to be removed.

Especially in the area of brain surgery, such as in resection of gliomas, the surgeon wants to resect the tumor tissue as thoroughly as possible while at the same time harming normal tissue as little as possible or not at all, to prevent any further neurological disturbance. Optimized resection based on an objective determination of the tumor borders is impossible with the methods known so far.

Known surgical microscopes are used as an aid in surgery and give the surgeon a magnified image of the area of the patient's body that is of interest while also offering other supporting features.

German Patent Reference DE 202005021111U1 discloses a combined diagnosis-supporting and therapy-supporting system. The system described there includes at least one light source, an image detection device, an image processing system and a projection system. Image information is detected by the image detection device and processed further in the image processing system. The image processing yields additional information which is made accessible to the surgeon so that this additional information is projected into the surgical area with the help of the projection system. The image may be projected by a beamer into the surgical area or into the eyepiece or the monitor of the surgical microscope, for example.

The surgeon sees the real image of the surgical field through the projection with a generated image superimposed on it, in which normal tissue appears with a green color, for example, or pathological tissue is marked by a higher intensity. This additional information is generated offline in the image processing system.

The document cited above describes the photodynamic diagnosis of tumor tissue on the basis of excitation of fluorescent radiation, but it does not disclose whether and how the image processing system is able to analyze the tissue portions to be removed or can determine and display incision lines, so that the surgeon can be supported by additional information in performing the resection.

The main emphasis of the disclosure of German Patent Reference DE 202005021111U1 is in the projection of additional information into the operating area, but it does not mention how the analysis, determination and calculation of additional information take place. According to the German Patent Reference cited above, those skilled in the art could not understand how normal tissue is to be differentiated from tumor tissue, which is why it is left up to the surgeon himself to evaluate the intensity of the fluorescence and/or the projected image and to determine which tissue is the tumor tissue that is to be removed. Since the visual perception and assessment of the fluorescence depend greatly on the particular surgeon, the extent of resection may vary considerably in some cases.

To prolong the time until recurrence of relapsing tumors, it is absolutely essential to remove as many tumor cells as possible.

Because color perception varies from one surgeon to the next and the subjective perception of a fluorescent image depends on the environment and lighting in the operating room, no objective method of determining tumor borders independently of the surgeon has thus been disclosed.

### SUMMARY OF THE INVENTION

One object of this invention is to provide a method which allows a quantifiable objective and reproducible determination of the borders of tumor tissue so that the subjective perception and the experience of the surgeon play no role.

The inventive method achieves this object and provides the associated support in resection of tumor tissue, so that only a minimal amount of normal tissue adjacent to the tumor is removed and thus additional neurological disorders can be better prevented.

Another object of this invention is to improve the quality of life and to increase the life expectancy of patients through virtually complete tumor removal that can be achieved.

### BRIEF DESCRIPTION OF THE DRAWINGS

One exemplary embodiment of the subject matter of this invention is described below in view of the attached drawings, wherein:

FIG. 1a shows a schematic diagram of a fluorescence microscope used as a surgical microscope;

FIG. 1b shows a graph of a fluorescence spectrum, where the intensity of the radiation is plotted as a function of the wavelength and the fluorescence excitation is visible;

FIG. 2 shows a fluorescent image of an illuminated and reflecting operating area;

FIG. 3 shows a fluorescent image of the illuminated operating area of FIG. 2 with superimposed critical limit intensity lines;

FIG. 4 shows a fluorescent image corresponding to FIG. 3, wherein areas of other intensities are delineated by additional contour lines;

FIG. 5 shows a simple contour plot of the limit intensity line, all black, and the additional contour lines generated by the image processing system; and

FIG. 6 shows a false-color image of the operating field with different contour lines and regions of different colors.

### DETAILED DESCRIPTION OF THE INVENTION

After a substance for generating a tumor detection feature, preferably 5-aminolevulinic acid has been administered orally to a glioma patient to generate a protoporphyrin fluorescence, the patient has been prepared for surgery, anesthetized and a surgical area 20, where the operation is to be performed has been made accessible, resection of tumor tissue 22 is performed using a surgical microscope.

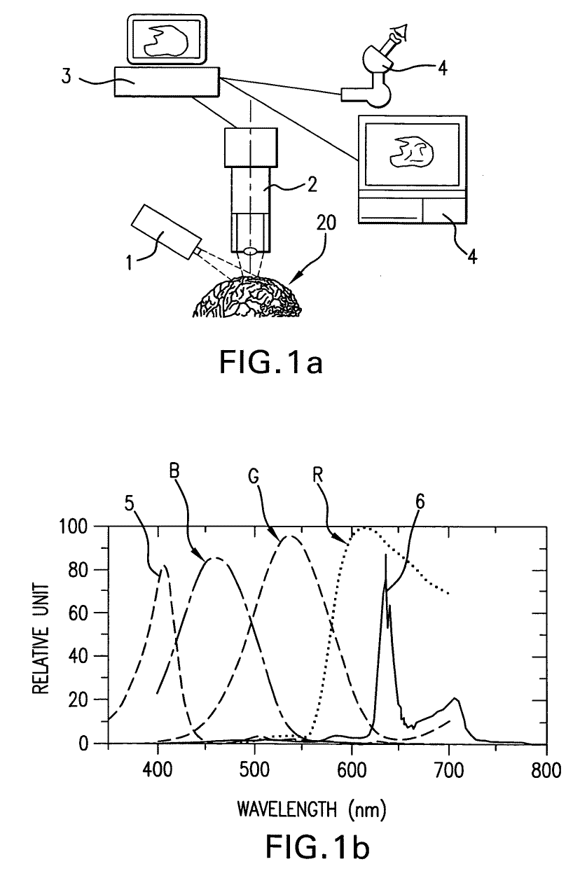

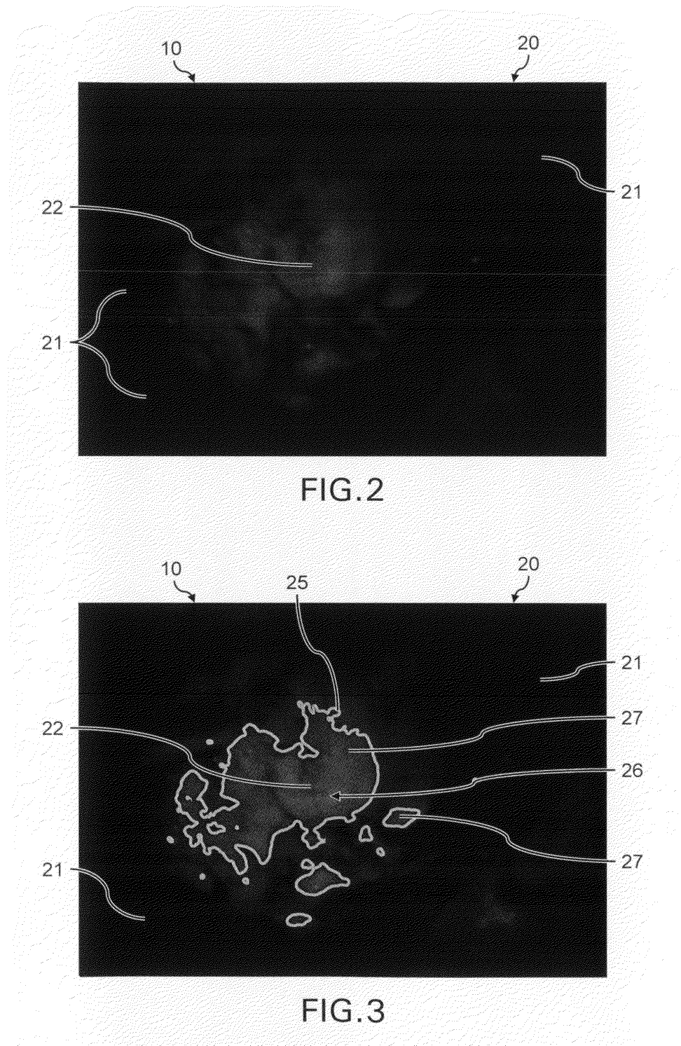

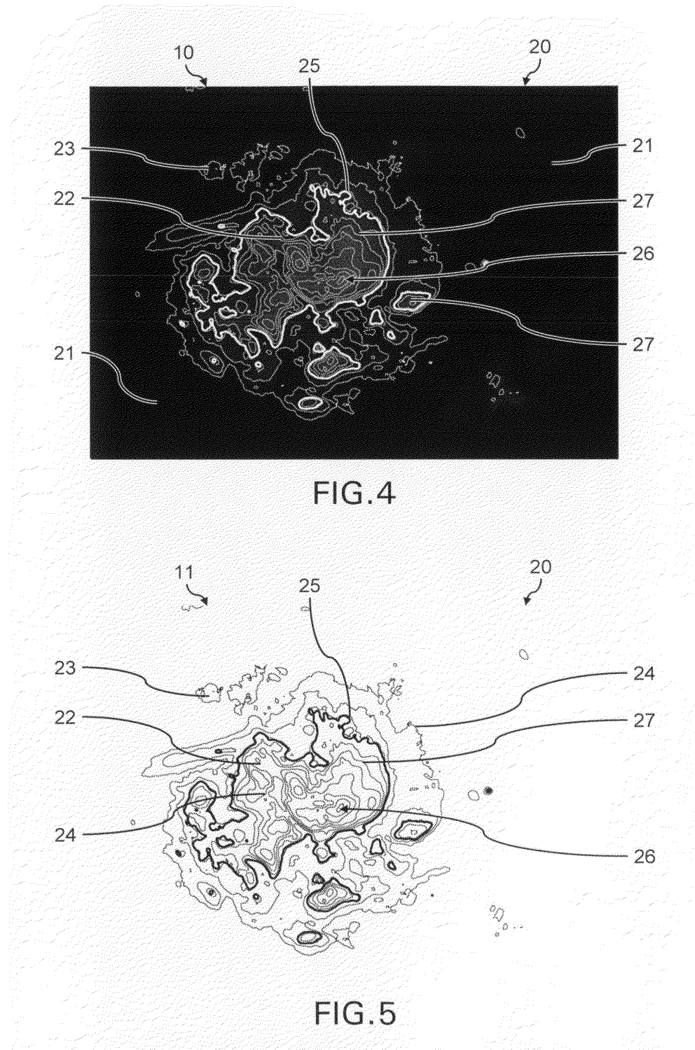

One possible arrangement of the surgical microscope is diagrammed schematically in FIG. 1a. The operating area 20 is illuminated with UV radiation, which is in the wavelength range of approx. 400 nm and thus is in the visible blue wavelength range, from an excitation device 1. The fluorescent UV radiation may originate from a xenon light source that has filters or from a laser, for example. The 5-aminolevulinic acid synthesizes protoporphyrin IX, which has fluorescent properties and which accumulates selectively in pathologically altered cells.

In addition to an excitation line 5 of the fluorescence-exciting UV radiation of the excitation device 1 at approx. 400 nm, the fluorescence spectrum of FIG. 1b also shows an emission line 6 of the emitted radiation of the fluorescent protoporphyrin IX in the visible spectral range from approx. 600 nm to 700 nm. The tumor tissue 22 fluoresces and emits red light according to emission line 6 in the visible spectral range, where the intensity of the emitted light correlates with the concentration of intracellular protoporphyrin IX.

An image detection device 2 collects and bundles the emitted light by optical components on a detector which generates digital image information in the form of a fluorescent image 10 transmitted from the surgical area 20. The actual detection in the image detection device 2 may be performed by a digital CCD camera or by a fluorescence spectrometer.

Due to the detection of the emitted radiation with a CCD camera that detects the intensities of the red component R, the green component G and the blue component B individually, then no additional filters are necessary in the range above 600 nm in the case of emission line 6 to obtain optimal measurement results. The great distance between the excitation line 5 and the emission line 6 is thus advantageous when using a CCD camera because the emission line 6 is situated or positioned only within the red component R and thus detection is not disturbed by the blue excitation line 5 in the blue component B. In addition, the quantum efficiency of most CCD sensors is greatest in a range of red light, and thus the highest sensitivity of a CCD camera is in the red range.

The image detection device 2 records various brightness values of the red component R, the green component G and the blue component B for each pixel. If an 8-bit sensor is selected, 256 different brightness values can be recorded. If a 16-bit sensor is selected, 65,536 different brightness values can be recorded and forwarded as a digital fluorescent image 10 directly to a display device 4, where they are displayed. The display device 4 may be a monocular or binocular eyepiece, a monitor or the like. The surgeon observes the entire procedure with the help of the display device 4.

In addition, the digital fluorescent image 10 is also forwarded to the image processing system 3. The image processing system 3 comprises a computer unit having at least one read/write memory and a computer program that executes the analysis and processing of the fluorescent images and accomplishes the output of generated line profile images 11 and generated false-color images 12 and superimposes the fluorescent image 10 on the generated images.

The fluorescence is in the red spectral range and the tumor tissue 22 enriched with protoporphyrin IX lights up red, so the red component R of the fluorescent image 10 is analyzed and processed to achieve a quantitative determination of the extents and limits of the tumor tissue 22. The first analysis step of the image processing system 3 is extraction of the red channel from the recorded fluorescent image 10.

The fluorescent image 10 has different regions of differing light intensities in the red spectral range. Optionally, the image processing system 3 or the surgeon determines the range in the fluorescent image 10 having a maximum intensity 26.

The image processing system 3 is able to ascertain the maximum intensity 26 by comparing the intensities of all pixels of the red component of the fluorescent image 10 and storing it as the maximum intensity 26. For the manual determination of the maximum intensity 26, an input device, such as a computer mouse, is connected to the image processing system 3 with which the surgeon selects the region of pixels in the fluorescent image 10 that in his opinion is the brightest. Whereas, an automatic determination of the maximum intensity 26 always determines reproducibly the intensity values that are in fact the highest as the maximum intensity 26, manual determination of the maximum intensity 26 by the surgeon prevents possible image errors in the fluorescent image 10 from being interpreted as the maximum intensity 26 by automatic determination. To prevent color blindness, if any, on the part of the surgeon from leading to problems in manual determination of the maximum intensity 26, it is advantageous for the red channel of the fluorescent image 10 of interest to be converted by known means into a gray-scale image before determining the maximum intensity 26 in the brightest region.

In a next step, a threshold value of intensity is defined by the image processing system 3, representing a fraction of the maximum intensity 26. The threshold value represents the intensity of the detected fluorescent radiation, which is between the intensity of normal tissue 21 and tumor tissue 22. As experiments have shown, a threshold value in the range of 30% of the maximum intensity 26 relatively accurately characterizes the threshold between healthy normal tissue 21 and tumor tissue 22.

Areas with pixels in the fluorescent image 10 having an intensity above this threshold value characterize tumor tissue 22, while tissue that emits light of an intensity above the threshold value, visible as an area in the fluorescent image 10, is classified as normal tissue 21. The desired threshold value may be stored in the image processing system 3 and may if necessary also be altered. Clinical experiments and analyses of surgeries that have already been performed with regard to the recurrence of tumor tissue 22 have confirmed a threshold value of 33% of the maximum intensity 26 to be particularly advantageous.

After determining the maximum intensity 26 and the threshold value, a binary image is generated, wherein pixels of the R channel of the fluorescent image 10 with intensities below the threshold value and pixels with intensities above the threshold value are differentiated. The borders between the regions with intensities greater than the threshold value and regions with intensities lower than the threshold value are determined by conventional methods of digital image processing and emphasized. By basic morphological operations of image processing, such as by dilatation and/or erosion with a structuring element, for example, in the form of a 3×3 matrix of ones, the borders between different image areas are determined. In practice, combinations and multiple applications of dilatation and erosion are advisable, such as opening and closing, so that edges are detected. By applying these known methods of digital image processing, image information including the border between tumor tissue 22 and normal tissue 21 is generated as limit intensity line 25, which is several pixels wide.

The width of the limit intensity line 25 is variable and is determined, among other things, by the structuring element which is a component of the morphological operators. The width of the limit intensity lines 25 may be varied if the structuring element is defined other than in the image processing system 3 accordingly. Other operations such as high-pass and low-pass filtering or gradient operator may be used to generate the limit intensity lines 25.

The limit intensity line 25 is self-contained and encloses a tumor tissue area 27 in which there are pixels with a higher intensity than the defined threshold value. The pixels in the tumor tissue areas 27 with intensities greater than the threshold value characterize tumor tissue 22. Outside of the enclosed limit intensity line 25, the pixels have intensities which are below the threshold value so that the tissue in the area of these pixels is defined as normal tissue 21 and the area is called the normal tissue area 23.

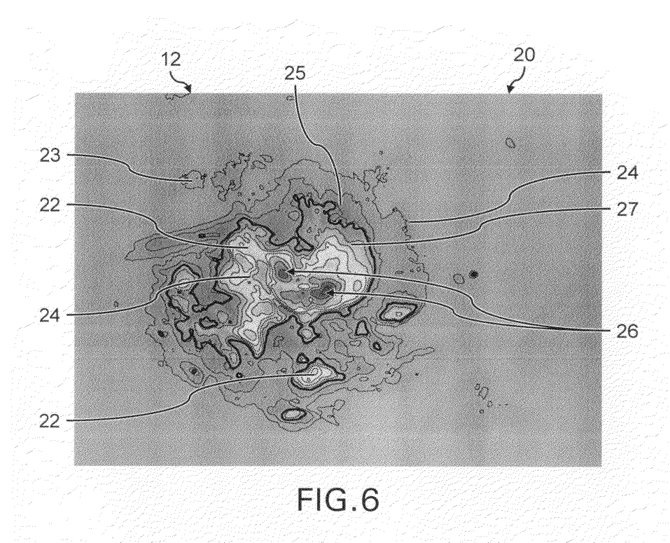

The image processing system 3 creates a line profile image 11 from the limit intensity lines 25 and this can be superimposed on the recorded fluorescent image 10 and can be imaged by means of the display device 4. FIG. 3 shows a fluorescent image 10 as an example, with a line profile image 11 according to FIG. 5 comprising several areas bordered by a closed limit intensity line 25 in each case superimposed on the fluorescent image. The line profile images 11 thereby generated may optionally be displayed separately, as shown in FIG. 5, without being superimposed on the fluorescent image 10, or as false-color image 12 as shown in FIG. 6 and as can be analyzed by the surgeon and selected for support in the surgery.

Whereas the digital fluorescent image 10 is displayed directly without processing in real time by the display device 4, line profile images 111 and false-color images 12 generated by the image processing system 3 may be superimposed on the fluorescent image 10 and displayed. Thus, the surgeon is able to see additional information about the surgical area 20 calculated and processed in his ordinary display device 4 and do so during the actual reception of the tumor tissue 22.

To analyze operations at a later point in time and optimize the threshold value determination by studies, measures are taken to store the recorded fluorescent images 10 and generated line profile images 11 and false-color images 12 for study purposes in the image processing system 3 on a hard drive or another read-only memory.

According to the method described above, it may be desirable for additional contour lines 24 to be displayed in addition to the limit intensity lines 25 already shown.

The contour lines 24 surround regions in the fluorescent image 10 whose intensities are a fixed defined distance from the defined threshold value. For example, intensity differences of 10% between the threshold value and the maximal intensity 26 may be selected. The contour lines 24 are shown in the line profile image 11 of FIG. 5. To differentiate the various contour lines 24, the contour lines 24 are represented differently by the display device 4, such as with dashed lines or dotted lines. Each contour line 24 surrounds an area of pixels whose intensity is in a certain ratio to the threshold value. It may be desirable for the contour lines 24 to surround normal tissue regions 23 and/or tumor tissue regions 27 with intensities above the threshold value up to the maximal intensity.

The regions enclosed by the contour lines 24, which are situated within a tumor tissue region 27 enclosed by a limit intensity line 25 characterize the various strongly fluorescent regions within the tumor tissue region 27. It may also be desirable to display normal tissue regions 23 outside of a tumor tissue region 22, which is surrounded by a limit intensity line 25, as bordered by a contour line 24. This is also shown in FIG. 5.

These additional contour lines 24 are also shown in the false-color image, or color-coded image 12 in FIG. 6, whereby to illustrate the different intensity of the fluorescent regions of the fluorescent image 10 which are colored with different colors from the tumor tissue regions 27 surrounded by the contour lines 24. The determination of the limit intensity lines 25 and the contour lines 24 and the coloring of the regions having different fluorescence are all performed by the image processing system 3. The surgeon may optionally display the fluorescent image 10 with the superimposed line profile 11 or the false-color image 12 or the line profile image 11 or the false-color image 12 with the fluorescent image 10 masked out.

To reduce individual errors in the determination of the extent of tumor tissue 22, the method described here is developed for quantified, objective and reproducible determination of the borders of tumor tissue 22. The determination of the threshold value may be performed by the surgeon or may be defined by the image processing system 3.

During a surgery, fluorescent images 10 are recorded continuously and the different regions analyzed on the basis of the maximal intensity 26 determined at the beginning and the threshold value, and the limit intensity lines 25 and the contour lines 24 are recalculated constantly and displayed. The tumor tissue regions 27 which are bordered by the limit intensity lines 25 are also reduced in area in the course of the operation, so that the tumor tissue 22 is reduced by completely resecting the tumor tissue 22. As soon as radiation with an intensity above the threshold value is no longer detected, then all the tissue classified as tumor tissue is removed and the surgery is concluded.

Swiss Patent Reference 01969/07, filed on 19 Dec. 2007, the priority document corresponding to this invention, and its teachings are incorporated, by reference, into this specification.

What is claimed is:

1. A method for analyzing and processing fluorescent images of a medical surgical microscope, comprising:
irradiating a surgical area (20) with UV radiation from an excitation device (1);
an image detection device (2) recording and forwarding a digital fluorescent image (10) to an image processing system (3), wherein a red channel (R) is extracted from the fluorescent image (10), and then defining a maximum intensity (26) and then determining a threshold value as a percentage amount of the maximum intensity (26);
determining limit intensity lines (25) which surround tumor tissue regions (27) whose pixels have intensities above a defined threshold value, wherein then a line profile image (11) is calculated which includes the limit intensity lines (25) of the tumor tissue regions (27) and is superimposed on the fluorescent image (10) in a display device (4), wherein regions not surrounded by limit intensity lines (25) are defined as normal tissue regions (23).

2. The method for analyzing and processing fluorescent images according to claim 1, wherein the threshold value is at least approximately 30% of the maximum intensity (26).

3. The method for analyzing and processing fluorescent images according to claim 2, wherein the threshold value is 33% of the maximum intensity (26).

4. The method for analyzing and processing fluorescent images according to claim 1, wherein the maximum intensity (26) is determined automatically by the image processing system (3).

5. The method for analyzing and processing fluorescent images according to claim 1, wherein the maximum intensity (26) is determined manually by a surgeon using an input device connected to the image processing system (3), wherein brightest pixels of the fluorescent image (10) are selected manually.

6. The method for analyzing and processing fluorescent images according to claim 1, wherein the red channel (R) of the fluorescent image (10) is converted into a binary image before determining the limit intensity lines (25) by the image processing system (3).

7. The method for analyzing and processing fluorescent images according to claim 6, wherein the limit intensity lines (25) are determined by morphological basic operations of image processing, including by dilatation and/or erosion with a structuring element which represents borders between tumor tissue (22) and normal tissue (21).

8. The method for analyzing and processing fluorescent images according to claim 7, wherein the structuring element of dilatation and/or erosion is a 3×3 matrix of ones.

9. The method for analyzing and processing fluorescent images according to claim 8, wherein a width of the limit intensity lines (25) is variable.

10. The method for analyzing and processing fluorescent images according to claim 6, wherein the limit intensity lines (25) are generated by high-pass filtering and/or low-pass filtering or by employing a gradient operator.

11. The method for analyzing and processing fluorescent images according to claim 1, wherein the image processing system (3) generates closed contour lines (24) from the red channel (R) of the fluorescent image (10) which have a fixed defined distance from the defined threshold value and represent the limits between areas of different intensities above and below the threshold value.

12. The method for analyzing and processing fluorescent images according to claim 1, wherein a false-color image (12) is displayed by the display device (4), showing the normal tissue regions (23) and the tumor tissue regions (27) in different colors.

13. The method for analyzing and processing fluorescent images according to claim 12, wherein the line profile image (11) and/or the false-color image (12) are superimposed on the fluorescent image (10) in an activatable and deactivatable manner.

14. The method for analyzing and processing fluorescent images according to claim 13, wherein a computer program product for processing fluorescent images of a medical surgical microscope has program parts for implementing a method, wherein a computer program product performs the steps of:
extracting the red channel (R) of the fluorescent image, determining the maximum intensity (26), determining a defined threshold value of the intensity as a fraction of the maximum intensity (26), generating the limit intensity lines (25) by basic morphological operations of image processing, including by dilatation and/or erosion with a structuring element, generating a line profile image (11) on a basis of the generated limit intensity lines (25), and superimposing the line profile image (11) on the fluorescent image (10) thus recorded.

15. The method for analyzing and processing fluorescent images according to claim 1, wherein a computer program product for processing fluorescent images of a medical surgical microscope has program parts for implementing a method, wherein a computer program product performs the steps of:
extracting the red channel (R) of the fluorescent image, determining the maximum intensity (26), determining a defined threshold value of the intensity as a fraction of the maximum intensity (26), generating the limit intensity lines (25) by basic morphological operations of image processing, including by dilatation and/or erosion with a structuring element, generating a line profile image (11) on a basis of the generated limit intensity lines (25), and superimposing the line profile image (11) on the fluorescent image (10) thus recorded.
